(19) United States
(12) Patent Application Publication
(10) Pub. No.: US 2024/0165321 A1
(43) Pub. Date: May 23, 2024

(54) Degassed Infusion System for Arterial Infusion

(71) Applicant: Zaid Aljuboori, Wilkes Barre, PA (US)

(72) Inventor: Zaid Aljuboori, Wilkes Barre, PA (US)

(21) Appl. No.: 18/165,728

(22) Filed: Feb. 7, 2023

Related U.S. Application Data
(60) Provisional application No. 63/384,416, filed on Nov. 19, 2022.

(51) Int. Cl. A61M 5/14; A61M 39/20

(52) U.S. Cl. CPC A61M 5/1411; A61M 39/20; A61M 2005/1403

(57) ABSTRACT

A system for arterial infusion provides integrally attached IV bag and multiple IV delivery lines pre-charged with degassed saline solution and sealed by capping the distal ends of the IV lines. Bubble-free arterial infusion may thus be quickly obtained by selecting an individual line, removing its cap, and connecting it to an appropriate needle or catheter.

### CROSS REFERENCE TO RELATED APPLICATION

This application claims the benefit of U.S. provisional application 63/384,416 filed Nov. 19, 2022, and hereby incorporated by reference.

### STATEMENT REGARDING FEDERALLY SPONSORED RESEARCH OR DEVELOPMENT

n/a

### BACKGROUND OF THE INVENTION

The present invention relates to an apparatus for the infusion of liquids during medical procedures and, in particular, to a multiline infusion system greatly reducing the risk of air bubbles during arterial infusion.

Angiography is a minimally invasive diagnostic procedure for the evaluation of a blood vessel, for example, coronary arteries, which is one of the most common operations performed in the United States. Moreover, it is commonly used to evaluate head and neck vessels as well as peripheral (upper and lower extremity) vessels. Also, angiographic findings such as presence of vascular malformation, vessel occlusion, injury, stenosis, etc., may necessitate an endovascular intervention such as unblocking of a vessel, occlusion of bleeding vessel, and others. These procedures involve obtaining arterial access then introducing arterial sheaths, catheters, and other devices into the artery. Each of these equipment requires constant flow of sterile normal saline through the sheath or catheter, with or without a medication (e.g., heparin), delivered using a bag and tubing that is connected to these sheaths and catheters. The fluid will be running continuously throughout the procedure to minimize the risk of forming blood clots that can result in significant complications.

Arterial infusions can cause arterial gas embolism (AGE) resulting from air bubbles in the infused liquid and within the bag and tubing. AGE is more serious than air bubbles introduced into a vein because the arteries lead directly to tissue that can be damaged when blood flow is interrupted. Symptoms of AGE include stroke, limb ischemia, and heart attack.

Air bubbles arise from dissolved gas in the saline solution precipitated by changes in surface tension as well as from residual undissolved air that doesn't dissolve in the infused liquid, especially nitrogen. To make the infused medicament bubble free, first the operator must connect the IV tube to the IV bag. Then, different techniques are used such as continuously running the fluid through the tubes while inspecting them and/or mechanically mobilizing the bubbles by flicking the tubes. This process is time consuming and costly as it can result in a significant waste in procedural time and human resources. Of note, the national average of operating room time cost in the United States is about $60 per minute. Moreover, Some IV bags and tubes end up being discarded because bubbles remain within the tube after reasonable effort.

The current operational definition of a bubble free system indicates that the tubing has no bubbles when seen by the naked eye. This does not mean the system is gas free, as gas particles remain in the system and can form bubbles when the surface tension changes.

### SUMMARY OF THE INVENTION

The present invention provides a sterile degassed fluid delivery system that is pre-filled during manufacture with degassed medicament and that provides multiple, capped attached lines permitting additional feeds of bubble-free medicament to be obtained on demand with minimal effort.

In one embodiment, the invention provides an arterial fluid delivery system having a medicament container providing a container volume communicating with a manifold joined to a set of at least two flexible tubes. The tubes are fixedly attached to the manifold at their proximal ends while a cap releasably seals the distal ends of the flexible tubes to define a system volume sealed against outside air. The system volume is filled with a degassed saline solution to the exclusion of bubble-forming gas.

It is thus a feature of at least one embodiment of the inventions to provide an infusion system that greatly reduces the risks, costs, and delays associated with providing arterial infusion. By eliminating the need for assembly, degassing, and monitoring for bubbles at the time of the procedure, wasteful diversion of medical personnel to these tasks is reduced and an immediate availability of infusion liquid at a moment's notice is obtained.

The saline solution may further include heparin.

It is thus a feature of at least one embodiment of the invention to provide an infusion system well adapted to procedures such as angiography.

The cap may provide a capping manifold joining the distal ends of each of the flexible tubes to a common manifold chamber sealed against outside air.

It is thus a feature of at least one embodiment of the invention to provide a cap to the infusion lines that can reduce tangling by holding the ends of multiple lines fixed with respect to each other and that can perform double duty during the manufacturing process to provide parallel flushing of the lines.

The capping manifold may include an irreversibly sealable manifold outlet adapted for flushing the lines during manufacture.

It is thus a feature of at least one embodiment of the invention to allow the manifold to be used for flushing during manufacture without promoting the introduction of air by the user through the outlet.

The medicament container may provide a sealable flush channel positioned in opposition to a communication between the container volume and the manifold at a top end of the medicament container as defined by normal operation of the intra-arterial delivery system.

It is thus a feature of at least one embodiment of the invention to provide an IV bag that permits flow-through flushing to scavenge air from the bag during the manufacturing process.

The delivery system may include a drip chamber positioned along each flexible tube, the drip chamber providing: an enclosed volume communicating with a drip spout inlet receiving medicament into the drip spout extending into the chamber volume to form drips of medicament that may fall through gas in the chamber volume; a drip chamber outlet collecting the drips of medicament to conduct them to a corresponding flexible tube; and a chamber flush inlet for receiving a flushing medium for scavenging gas from the drip chamber.

It is thus a feature of at least one embodiment of the invention to provide a drip chamber that can be effectively flushed with a high flow rate of fluid that would otherwise be unduly constrained by the drip spout.

The chamber flush inlet may join with the drip chamber volume at an uppermost end of the drip chamber volume as defined by normal operation of the drip chamber.

It is thus a feature of at least one embodiment of the invention to provide a drip chamber design without blind recesses that could trap air bubbles.

The enclosed volume of the drip chamber may further communicate with a vent opening closed by a resealable cap to allow venting of air into the drip chamber during use.

It is thus a feature of at least one embodiment of the invention to allow the drip chamber to be shipped filled with medicament to prevent gas from dissolving into the medicament during shipping while allowing the introduction of a necessary volume of gas immediately before use necessary for the functioning of the drip chamber. The inventor has determined that the short period of time after introduction of this air and the quiescent state of the medicament avoids significant introduction of air into the medicament.

The distal ends of the tubes may provide luer locks engaging the caps for subsequent attachment to intra-arterial devices (e.g., sheath, catheters, and others).

It is thus a feature of at least one embodiment of the invention to provide a capping of the tubes that can employ a standard connector for catheters and the like, thus greatly simplifying the task of removing and connecting a line to a catheter or other device for use while sealing the line prior to use.

The delivery system may further include a pair of metering clamps on each flexible tube operative to control flow through the tube.

It is thus a feature of at least one embodiment of the invention to allow closure of the tube near the distal end during removal to reduce the introduction of air during removal and attachment to a catheter or the like.

The invention also includes a method of manufacturing this device by circulating degassed water through the flexible tubes and medicament container to remove air therefrom; removing the degassed water to fill the system volume with saline solution; and sealing the caps to the distal ends of the tubes.

It is thus a feature of at least one embodiment of the invention to provide an infusion system having superior degassing possible during manufacture.

The degassing may further include a flushing the system with carbon dioxide.

It is thus a feature of at least one embodiment of the invention to provide a method of excluding air using a gas that readily dissolves in water and thus that can be removed with a subsequent water rinsing.

The medicament container may further provide a sealable flush channel positioned in opposition to the opening between the medicament container and the manifold, and the flushing may be along a path through the container volume from the sealable flush channel to the manifold through a central portion of the container volume.

Thus, it is a feature of at least one embodiment of the invention to provide a mechanism for removing air from the medicament container by a rapid flushing flow of fluid.

The intra-arterial fluid delivery system may further include a drip chamber having a drip spout inlet receiving medicament and a chamber flush inlet for receiving a flushing medium for scavenging gas from the drip chamber; and the flushing may be along a path providing parallel flow through both the drip spout and the chamber flush inlet.

The cap of the IV lines may be a manifold joining the distal ends of each of the flexible tubes to a common manifold chamber sealed against outside air via a sealable manifold outlet; and the flushing is along a path between the lines and the manifold and through a sealable manifold outlet, after which the manifold outlet is sealed.

It is thus a feature of at least one embodiment to provide for a parallel flushing of all lines through the use of a manifold that can also serve to corral the lines during shipping.

These particular objects and advantages may apply to only some embodiments falling within the claims and thus do not define the scope of the invention.

### BRIEF DESCRIPTION OF THE DRAWINGS

FIG. 1 is a fragmentary, elevational view of one embodiment of the intra-arterial fluid delivery system as oriented during normal use showing an IV bag, manifold, multiple lines drip chambers and metering devices, and a distal sealing capping device.

FIG. 2 is an expanded cross-sectional, elevational view of a drip chamber showing a vent opening, flush opening, drip spout, and outlet port.

FIG. 3 is a process diagram showing a preparation of the intra-arterial fluid delivery system of FIG. 1 after assembly of the components and during a flushing and filling process.

FIG. 4 is a flow chart of the process of FIG. 3.

FIG. 5 is a figure similar to that of FIG. 1 showing an embodiment with a single IV line.

### DETAILED DESCRIPTION OF THE PREFERRED EMBODIMENT

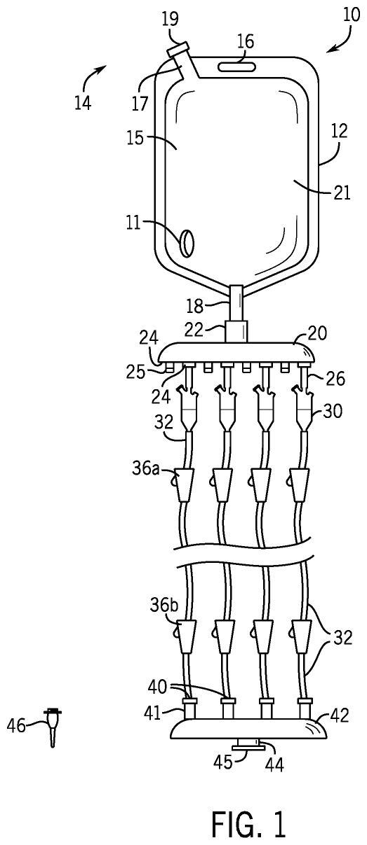

Referring now to FIG. 1, an intra-arterial fluid delivery system 10, suitable for use with angiography or other procedures in which fluid is introduced into the arteries, may provide for an IV bag 12 containing a medicament 15 such as a saline solution with or without heparin or other medications. As will be discussed below the medicament 15 will be degassed during manufacture.

The IV bag 12 may be made of a flexible, medical grade plastic such as polyvinyl chloride (PVC) and will typically have a volume 21 ranging from 100 mL to 5000 mL and in one embodiment greater than 500 ml. An upper end 14 of the IV bag 12, as defined by its normal use during an infusion, may provide a hanger opening 16 allowing the bag to be suspended from an IV pole (not shown) as is generally understood in the art.

A lower end of the IV bag 12 may provide for an outlet port 18 through which medicament may be delivered to a manifold 20. An optional drug introduction port 11 may be provided in the bag to allow drugs to be introduced into the medicament 15 by the user, the drug introduction port 11 providing an elastomeric opening that allows introduction of a hypodermic needle without the passage of air or the like around the needle.

A flushing port 17 may be provided at the upper end 14 of the IV bag 12 communicating with the volume 21 and sealable with a cap 19. The cap 19 is designed to be readily attached to the flushing port 17 but then to resist accidental or unnoticed removal by an end-user. This may be accomplished, for example, by locking detents on a threaded coupling, adhesive, or other methods known in the art, which resist removal once the cap 19 is installed or by means of features that provide evidence of tampering if removal of the cap is attempted, for example, by the destruction of frangible elements that result in a visual indication of tampering. Caps of this kind will be termed “irreversibly sealable” herein, meaning that they can be readily sealed when open but resist undetectable and inadvertent unsealing by a user after first being closed.

The outlet port 18 of the IV bag 12 may be in fluid communication with an inner chamber of the manifold 20 through an irreversibly sealing coupling 22 allowing these elements to be assembled while resisting disassembly as discussed above. The locking coupling 22 may incorporate a standard spike attached to the manifold 20 for insertion into the IV bag 12 or may make use of a pre-existing channel in the IV bag 12 eliminating the need for a spike.

The internal chamber of the manifold 20 may also communicate with multiple outlet ports 24 positioned on a lower edge of the manifold 20. Some of the outlet ports 24 are irreversibly sealed during manufacture, as will be discussed below; however other outlet ports 24 provide fluid communication with short connector lines 26 joining the manifold 20 to the upper ends of corresponding drip chambers 30. The drip chambers 30 may be constructed of a biocompatible, rigid, and transparent material such as a molded thermoplastic material, for example, acrylonitrile butadiene styrene (ABS), and provide an enclosed chamber whose internal volume is visible from outside of the chamber as will be discussed below.

The lower end of the drip chambers 30 communicates with proximal ends of two or more IV lines 32, for example, constructed of tubes of PVC, polyethylene, or polypropylene plastic. Four IV lines 32 are shown; however, the invention may accommodate a wide range of different numbers of IV lines 32, in some embodiments providing three or more IV lines 32. Each IV line 32 will typically be in excess of 50 inches long and may exceed 100 inches long and have a lumen ranging from 14 gauge to 26 gauge.

The connections between the connector lines 26 and the manifold 20 and drip chambers 30 and the IV lines 32 are such as to prevent ready decoupling by a user, for example, by adhesive, barb fitting, ultrasonic welding, or the like.

Positioned along each of the IV lines 32 are upper and lower flow-metering devices 36a and 36b, for example, roller clamps, which allow the flexible IV lines 32 to be progressively compressed to control flow therethrough or to be blocked by complete compression using a roller camming against the IV line 32 as retained along an inwardly sloped track. Other well-known IV-line clamp systems may be provided.

The distal ends of the IV lines 32 may be attached to standard male luer lock type connectors 40 suitable for attachment to intra-arterial catheters or the like and, by means of these connectors 40, may be connected to a capping manifold 42 having mating compatible luer-lock connectors 41. The capping manifold 42 joins the IV lines 32 via a common shared volume which also communicates with an outlet port 44. The outlet port 44 is irreversibly sealable by a cap 45 so that the capping manifold 42 effectively block the distal ends of the IV lines 32.

The capping manifold 42 operates in conjunction with the other above-described components to define a system volume being the inter-communicating volumes of the bag 12, manifold 20, connector lines 26, drip chambers 30, IV lines 32, and manifold 42. After manufacture but prior to use, this system volume is sealed against infiltration of outside air that might introduce bubbles into the contained degassed medicament 15 which fills the system volume to the exclusion of bubble-forming gas.

During use, a connector 40 for one or more of the IV lines 32 may be removed from the manifold 42 after locking of the flow-metering device 36b positioned at the distal end of that line and may be attached to a catheter 46 or other similar device having a female luer lock connection. A small amount of medicament can then be flushed through the catheter or other device by opening the flow-metering devices 36b prior to insertion or connection of the device to the patient's arteries and introduction of medicament 15 into the patient thus providing the benefits of a fully degassed medicament 15 with minimal effort.

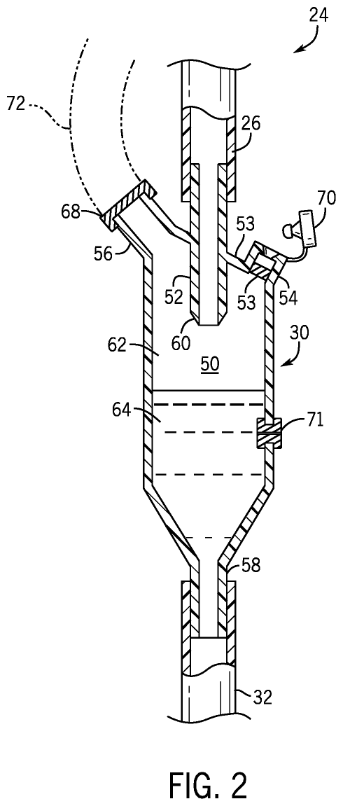

Referring now to FIG. 2, each of the drip chambers 30 may provide an enclosed drip chamber volume 50 communicating with a drip spout 52, an air vent 54, a flushing tube 56, and an outlet port 58. The drip spout 52 will project downwardly from an upper drip chamber wall 53 into the volume 50 to terminate at a drip nozzle 60 sized to promote the formation of medicament drops of uniform size. These drops can be observed and counted through the walls of the drip chambers 30 as they pass through an air space within the drip chamber 30 to a pool 64 of medicament 15 at the bottom of the drip chamber 30. The medicament 15 may then exit through the outlet port 58 at the bottom of the drip chamber 30. The drip spout 52 also extends upwardly through the upper drip chamber wall 53 to be attached to the tubes 28 to receive medicament therefrom.

The upper drip chamber wall 53 may slope upwardly to the flushing tube 56 so that the latter is positioned at a high point within the drip chamber volume 50 (a low point when the drip chamber 30 is inverted), and the walls of the drip chamber are constructed to eliminate blind pockets that would trap air in either of these positions so that flushing liquid passing through the flushing tube 56 can better scavenge gas from the drip chamber volume 50 during a flushing process. For this purpose, the flushing tube 56 will provide a greater flow rate than the constricted drip chamber spout 52, for example, at a given pressure, for example, 1.5 times the flow rate, and will typically have a greater lumen cross-sectional area, for example, by at least 50%. As shipped, the flushing tube 56 will be closed with an irreversibly sealable cap 68.

The drip chamber 30 also provides an air vent 54 desirably located in the upper portion of the drip chamber volume as oriented during normal use so as to admit air to the upper region 62 above the pool 64. This air, introduced contemporaneous with first use of the fluid delivery system 10, will not have time to dissolve into the medicament 15 in meaningful amounts. The air vent 54 may provide for a filter 55 sized to prevent dirt and bacteria from entering the volume 50 during use and will have a releasable cover 70 that can be removed to allow air to pass into the air vent 54 for proper adjustment of the height of the pool 64 and then snapped back into place to block further such airflow during use. Before opening of the air vent 54 by the end-user, the volume 50 will be wholly filled with degassed medicament 15. Generally, the size of the opening through the air vent 54 will be such as to allow air to enter the volume 50 at a faster rate than medicament 15 can pass through the spout 52 to allow the level of the pool 64 to be lowered despite the flow of medicament 15 from the bag 12.

Alternatively, an optional aspiration port 71 may be provided through a wall of the drip chamber 30 allowing the introduction of a hypodermic needle or the like through a self-sealing plug to draw fluid from the volume 50 while air passes inwardly through the air vent 54 eliminating the need to create the necessary air volume by flow of medicament 15 through the outlet port 58. The height of the aspiration port 71 may serve to define the desired height of the air pocket, preventing further liquid withdraw when the liquid level drops below the port 71.

In an alternate embodiment the functions of the air vent 54 and flushing tube 56 may be combined. In this construction, the cap 68 is replaced by the snap-in vent cover 70 and the filter 55 is removable or omitted during flushing to be described.

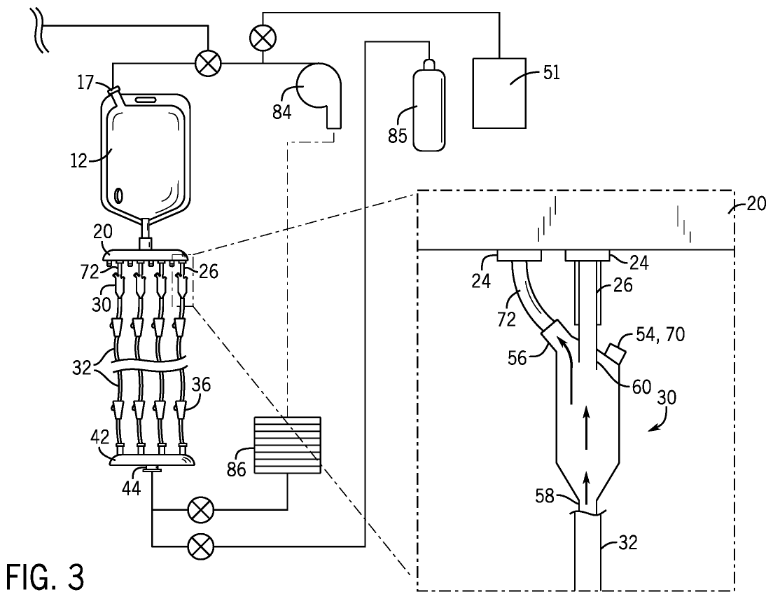

The above description is of the inter-arterial fluid delivery system 10 as manufactured and before and during use. Referring now to FIG. 3, the intra-arterial fluid delivery system 10 may be manufactured by assembly together with the bag 12, manifold 20, connector lines 26, drip chambers 30, IV lines 32, and the capping manifold 42 as discussed above. In addition, flushing connector tube 72 is installed between the manifold 20 and the drip chambers 30 as will be discussed below.

In one embodiment, the intra-arterial fluid delivery system 10 may be oriented with the bag 12 in the uppermost position consistent with its normal orientation during use; however, an inverted configuration is also possible. In this first configuration, the upper surfaces of the manifolds 20 and 42 present a continuous downward concave surface to prevent entrapment of gas in blind passageways. Such entrapment is also avoided by the design of the top wall 53 of the drip chambers 30 discussed above. As noted, both the drip spout 52 and the flushing tube 56 are connected to the manifold 20 via outlet ports 24 to allow a high flow rate through the drip chamber 30 to scavenge air from the drip chamber 30 using connector lines 26 from the drip nozzle 60 to the manifold 20 and flushing connector tube 72 and from the flushing tube 56 to the manifold 20.

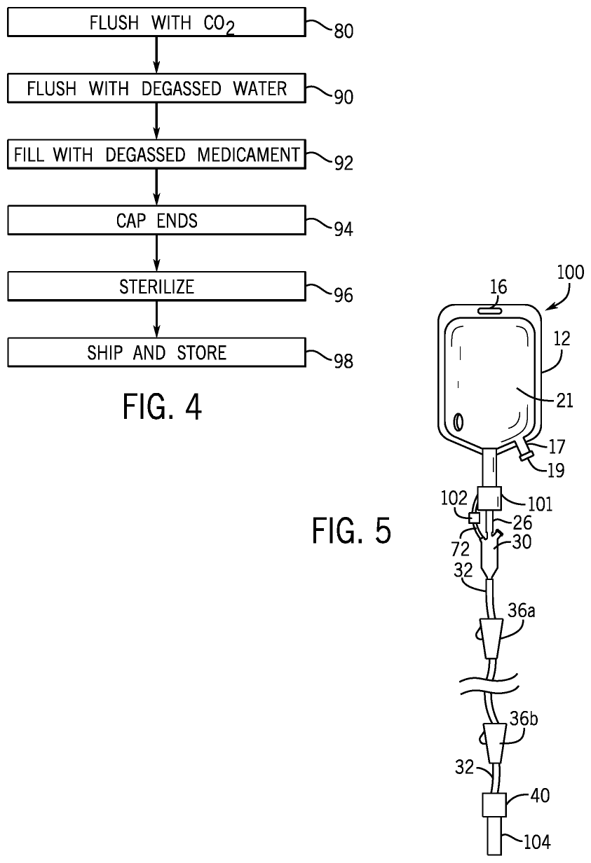

Referring now also to FIG. 4, as indicated by process block 80, in a first step after assembly, the system volume is flushed with dry carbon dioxide from a tank 82 flowing upwardly through the outlet port 44 (with the cap 45 not yet installed) and upwardly through the manifold 42 and through each of the IV lines 32 (with the flow-metering devices 36 in an open position). The carbon dioxide desirably free from nitrogen and other gases, for example, having a purity of greater than 99%. The gas then continues through the drip chambers 30 exiting via the drip nozzle 60 and flushing tube 56 into the manifold 20 upward into the bag 12 and out of the flushing port 17 (with the cap 19 not yet installed). The carbon dioxide removes residual air and in particular nitrogen from within the system.

After this flushing process, a valve to the carbon dioxide tank 85 is closed and chilled, degassed water or other degassed fluid is circulated by pump 84 upwardly through the outlet port 44 along the same path in a closed loop also through a degassing assembly 86, for example, using membrane degasification, ultrasonic gassing, sparging, or the like. Any residual CO₂ is dissolved in this water and thus removed.

At the conclusion of this rinsing process and as indicated by process block 92, the system is filled with degassed medicament 15 by appropriate switching of valves to completely fill the system volume displacing any gas that might infiltrate.

After filling, the flushing connector tube 72 may be removed and the caps 68, 19 and 45 installed together with covers 25 on the outlets 24 as necessary and the flow-metering devices 36 closed.

The entire assembly is then sterilized as indicated by process block 96 and shipped per process block 98.

When received at a medical facility, a fully degassed intra-arterial fluid delivery system 10 is available for immediate use with the degassed medicament made ready by simply venting, through air vent 54, a small amount of air necessary to establish the air pocket in the volume 50 (shown in FIG. 2) and removing one or more connectors 40 from the manifold 42 (shown in FIG. 1) attaching them to needles or the like for immediate use after opening the flow-metering devices 36.

The invention contemplates that alternative fluids may be used for degassing in place of the degassed water, for example, ionic liquids and mixtures with or without vacuuming. Moreover, several emerging technologies (e.g., distillation, extraction, adsorption, membrane separation, aqueous two-phase extraction) that potentially can be used in isolated or combined manner to recover the ionic liquids after their use. This recycling process can reduce the cost and environmental effects. Generally, it is desirable that the liquid be a good solvent for gas, for example, having a Henry's constant of less than 100 (L atm/mol) for carbon dioxide at room temperature or better than that of water.

It will be appreciated that the manifold 20 as described above may be wholly or in part integrated into the bag 12 and that multiple IV bags 12 may be attached to a manifold 20 for manufacturing convenience. Desirably 4-5 L of liquid will be available. It will be appreciated from this discussion that a single IV bag of selected volume may be attached to the upper manifold, or multiple IV bags can be connected to the upper manifold.

Referring now to FIG. 5, a reduced cost version of the present invention may provide an integrated single-line IV system 100 in which the IV bag 12 attaches through a T-coupling 101 with lines 26 and 72, the latter line 72 used during manufacturing as discussed before and having a pinch clamp 102 to close it off after manufacturing. Desirably, again, the inter-attachment of the tubes 26 and 72 and the IV bag 12 to the T coupling 100 will be via irreversible sealing as discussed above. The connector 40 may attach to a luer lock cap 104 installed during manufacturing to prevent the ingress of air and removed prior to use. The manifolds 20 and 42 may thus be eliminated. An alternative location of the flushing port 17 is shown providing more convenient manufacturing and which may be accommodated by sufficiently high flow rates of degassed water to ensure a thorough scouring of the entire inner volume 21 of the IV bag 12. Other features of this version will comport with the descriptions provided above.

As used herein, the term “degassing” refers to removal of dissolved gases to a degree that would prevent bubble formation in the liquid at room temperature sufficient to create a risk of air embolism if transferred into an artery, being generally amount of less than 0.5 mL of gaseous state air. Degassing may be distinguished from creating a system which is visually “bubble-free” and addresses a potential for bubble formation for example during storage or shipping. Degassing as used in this application requires that the system (IV bag, IV tubing, and medicament) be processed to remove gas, typically using a technique specifically directed toward that goal, for example, using membrane degasification, ultrasonic degassing, sparging, vacuum degassing, or the like. A degassed fluid will have less than 3% dissolved gas and preferably less than 1% dissolved gas and in practical cases less than 0.5% dissolved gas.

The term “saline” as used herein refers to solutions of water and sodium chloride suitable for intravenous use including without limitation: normal saline, Ringer's lactate solution, acetated Ringer's solution, as well as intravenous sugar solutions such as 5% dextrose in normal saline (D5NS), 10% dextrose in normal saline (D10NS), 5% dextrose in half-normal saline (D5HNS), and 10% dextrose in half-normal saline (D10HNS) as well as saline solutions containing other medicines where saline is the primary component.

The terms “IV line” and “IV bag” are used according to their ordinary meaning to qualify a type of line and type of bag suitable for medical infusions and are not intended to be limited to infusions into veins as might otherwise be suggested by intravenous. The term “medicament” is used to indicate fluids that can be introduced into the body for medicinal purposes including sailing with or without other drugs, ringer lactate, or saline with different sodium concentrations.

Certain terminology is used herein for purposes of reference only, and thus is not intended to be limiting. For example, terms such as “upper”, “lower”, “above”, and “below” refer to directions in the drawings to which reference is made unless otherwise qualified. Terms such as “front”, “back”, “rear”, “bottom”, and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including”, and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention is not limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What I claim is:

1. An intra-arterial fluid delivery system comprising:
a medicament container providing a container volume of at least 100 ml.
a set of at least two IV tubes
a manifold communicating between the volume and the set of at least two IV tubes fixedly attached to the manifold at their proximal ends for the delivery of medicament from the volume intra-arterially from distal ends of the IV tubes,
at least one cap releasably sealing the distal ends of the IV tubes to define a system volume in fluid communication with the medicament container and the IV tubes and sealed against outside air: and a degassed saline solution filling the system volume to an exclusion of bubble-forming gas.

2. The intra-arterial fluid delivery system of claim 1 wherein the saline solution further includes heparin.

3. The intra-arterial fluid delivery system of claim 1 wherein the at least one cap is a capping manifold joining the distal ends of each of the IV tubes to a common manifold chamber sealed against outside air.

4. The intra-arterial fluid delivery system of claim 3 wherein the capping manifold includes an irreversibly sealable manifold outlet adapted for flushing the IV tubes during manufacture.

5. The intra-arterial fluid delivery system of claim 1 wherein the medicament container further provides a sealable flush channel positioned in opposition to a communication between the container volume and the manifold at a top end of the medicament container as defined by normal operation of the intra-arterial delivery system.

6. The intra-arterial fluid delivery system of claim 1 further including a drip chamber positioned along each IV tube, the drip chamber providing an enclosed volume communicating with a drip spout inlet receiving medicament into the drip spout extending into the chamber volume to form drips of medicament that may fall through gas in the chamber volume; a drip chamber outlet collecting the drips of medicament to conduct them to a corresponding IV tube; and a chamber flush inlet for receiving a flushing medium for scavenging gas from the drip chamber.

7. The intra-arterial fluid delivery system of claim 6 wherein the chamber flush inlet provides for a greater flow rate at a given pressure than the drip spout inlet.

8. The intra-arterial fluid delivery system of claim 6 wherein the chamber flush inlet joins with the drip chamber volume at an uppermost end of the drip chamber volume as defined by normal operation of the drip chamber.

9. The intra-arterial fluid delivery system of claim 6 wherein the enclosed volume of the drip chamber further communicates with a vent opening closed by a resealable cap to allow venting of air into the drip chamber during use.

10. The intra-arterial fluid delivery system of claim 9 wherein the vent opening further includes a filter sized to prevent an introduction of bacteria into the drip chamber volume.

11. The intra-arterial fluid delivery system of claim 1 wherein the distal ends of the IV tubes provide luer locks engaging the at least one cap for subsequent attachment to intra-arterial catheters.

12. The intra-arterial fluid delivery system of claim 1 further including at least one metering clamp on each IV tube operative to control flow through the IV tube.

13. The intra-arterial fluid delivery system of claim 12 further including at least two metering clamps on each IV tube.

14. A method of manufacturing an intra-arterial fluid delivery system of a type having:
an IV bag providing a volume of at least 100 ml.
an IV tube fixedly attached to the IV bag at its proximal end for the delivery of medicament from the volume from a distal end of the IV tube.
a cap releasably sealing the distal end of the IV tube to define a system volume in fluid communication with the IV bag and IV tubes and sealed against outside air: and
a degassed saline solution filling the system volume to an exclusion of bubble-forming gas.
the method comprising in this order:
(a) flushing the system volume with scavenging liquid by circulating the scavenging liquid through the IV tubes and medicament container to remove air therefrom.
(b) filling the system volume with degassed saline solution; and
(c) sealing the at least one cap to the distal ends of the IV tubes.
where the scavenging liquid provides a Henry's constant of less than 100 (L atm/mol) for carbon dioxide at room temperature.

15. The method of claim 14 further including before (a) flushing the system volume with carbon dioxide.

16. The method of claim 14 wherein the medicament container further provides a sealable flush channel positioned in opposition to an opening between the medicament container and the IV tube and wherein the flushing is along a path from the sealable flush channel to the IV tube through a central portion of the container volume.

17. The method of claim 14 wherein the intra-arterial fluid delivery system further includes a drip chamber positioned along each IV tube, the drip chamber providing an enclosed volume communicating with a drip spout inlet receiving medicament into the drip spout extending into the chamber volume to form drips of medicament that may fall through gas in the chamber volume; a drip chamber outlet collecting the drips of medicament to conduct them to a corresponding IV tube; and a chamber flush inlet for receiving a flushing medium for scavenging gas from the drip chamber; and wherein the flushing is along a path providing parallel flow through both of the drip spout and the chamber flush inlet.

18. The method of claim 14 wherein the saline solution further includes heparin.
